(19) United States
(12) Patent Application Publication
(10) Pub. No.: US 2021/0330271 A1
(43) Pub. Date: Oct. 28, 2021

(54) MEDICAL IMAGING APPARATUS AND MEDICAL IMAGING PROCESSING METHOD

(71) Applicant: SAMSUNG ELECTRONICS CO., LTD., Suwon-si (KR)

(72) Inventors: Myung-sung SONG, Hwaseong-si (KR); Sung-hun PARK, Seoul (KR)

(73) Assignee: SAMSUNG ELECTRONICS CO., LTD., Suwon-si (KR)

(21) Appl. No.: 16/479,746

(22) PCT Filed: Jan. 12, 2018
(86) PCT No.: PCT/KR2018/000598

§ 371 Date: Jul. 22, 2019

(30) Foreign Application Priority Data
Jan. 23, 2017 (KR) 10-2017-0010678

(51) Int. Cl. A61B 5/00; A61B 5/055; G06T 3/40

(52) U.S. Cl. CPC A61B 5/7425; A61B 5/743; A61B 5/748; G06T 3/40; A61B 5/055

(57) ABSTRACT

Provided is a medical imaging apparatus including: a display; and a controller configured to obtain a plurality of scout images corresponding to planes that are perpendicular to one another, receive an input that selects a region of interest in a first scout image corresponding to a first plane from among the plurality of scout images, and display, in a second scout image corresponding to a second plane from among the plurality of scout images, a first quadrangle indicating a third plane that is a plane corresponding to the region of interest, based on the input.

The controller may display the first quadrangle so that at least a portion of the first quadrangle has a fade effect, and the fade effect may depict a level of darkness corresponding to a distance from the second plane.

### TECHNICAL FIELD

The present disclosure relates to a medical imaging apparatus and a method of processing a medical image, and more particularly, to a medical imaging apparatus for medical imaging planning and a method by which the medical imaging apparatus processes a medical image.

### BACKGROUND ART

Magnetic resonance imaging (MRI) apparatuses for capturing an image of an object by using a magnetic field are widely used to accurately diagnose disease because the MRI apparatuses three-dimensionally show not only bones but also disks, joints, nerves, ligaments, and the heart at desired angles.

In order to obtain a medical image of a region of interest, scout images may be first obtained so that an operator of a medical imaging apparatus selects an area to be imaged. The scout images may have a resolution less than that of a final image of the region of interest. Also, a time taken to obtain an image by using a scout scan may be less than a time taken to obtain the final image. Examples of the scout images may include images in an axial view, a sagittal view, and a coronal view.

The operator of the medical imaging apparatus may select the region of interest in the scout images by using a user interface through which the area to be imaged may be selected.

### DESCRIPTION OF EMBODIMENTS

### Technical Problem

Objectives of one or more embodiments are to three-dimensionally show a section of interest in a user interface for planning a medical image. Also, objectives of one or more embodiments are to improve spatial understanding of an operator who captures a medical image and provide a convenient medical imaging environment.

### Solution to Problem

A medical imaging apparatus according to an embodiment may include: a display configured to display a user interface; and a controller configured to obtain a plurality of scout images corresponding to planes that are perpendicular to one another, receive an input that selects, through the user interface, a region of interest in a first scout image corresponding to a first plane from among the plurality of scout images, and display, in a second scout image corresponding to a second plane from among the plurality of scout images, a first quadrangle indicating a third plane that is a plane corresponding to the region of interest, based on the input.

The controller may be further configured to display the first quadrangle so that at least a portion of the first quadrangle has a fade effect, and the fade effect may depict a level of darkness corresponding to a distance from the second plane.

The controller may be further configured to display, in a reduced shape, a second quadrangle corresponding to the first quadrangle on an outer portion of the second scout image.

The controller may be further configured to display the second quadrangle so that at least one side of the second quadrangle includes a guide line depicting depth perception.

The controller according to an embodiment may be further configured to: display the first quadrangle so that the first quadrangle overlaps a position indicating the region of interest in the second scout image, display a line of intersection between the second plane and the third plane on the second scout image; and display the first quadrangle so that only a portion closer to a point of view of the second scout image than the second plane has the fade effect.

The controller according to an embodiment may be further configured to display the first quadrangle so that at least one side of the first quadrangle includes a guide line depicting depth perception.

The controller according to an embodiment may be further configured to display a first line indicating the region of interest included in the first scout image.

The first line according to an embodiment may correspond to a line of intersection between the first plane and the third plane.

The first plane according to an embodiment may be perpendicular to the third plane.

Each of the plurality of scout images according to an embodiment may correspond to one from among an axial view, a sagittal view, and a coronal view.

The controller according to an embodiment may be further configured to obtain a magnetic resonance (MR) image of the region of interest.

A method of processing a medical image according to an embodiment may include: displaying a user interface; obtaining a plurality of scout images corresponding to planes that are perpendicular to one another; receiving an input that selects a region of interest through the user interface including a first scout image corresponding to a first plane from among the plurality of scout images; and displaying, in a second scout image corresponding to a second plane from among the plurality of scout images, a first quadrangle indicating a third plane that is a plane corresponding to the region of interest, based on the input.

In the method of processing the medical image according to an embodiment, the first quadrangle may be displayed so that at least a portion of the first quadrangle has a fade effect, wherein the fade effect depicts a level of darkness corresponding to a distance from the second plane.

### Advantageous Effects of Disclosure

According to embodiments, a medical imaging apparatus may three-dimensionally show a section of interest through a user interface for planning a medical image. Accordingly, the medical imaging apparatus may improve spatial understanding of an operator who plans the medical image and may provide a convenient medical imaging environment.

### BRIEF DESCRIPTION OF DRAWINGS

FIG. 1 is a block diagram of a medical imaging apparatus (e.g., a magnetic resonance imaging (MRI) apparatus), according to an embodiment.

FIG. 2 is a cross-sectional view for explaining images obtained by the medical imaging apparatus, according to an embodiment.

FIG. 3 is a flowchart of a method by which the medical imaging apparatus processes a medical image, according to an embodiment.

FIG. 4A illustrates a first scout image of an object displayed through a user interface by the medical imaging apparatus, according to an embodiment.

FIG. 4B illustrates a second scout image of the object displayed through the user interface by the medical imaging apparatus, according to an embodiment.

FIG. 4C illustrates a second scout image of the object displayed through the user interface by the medical imaging apparatus, according to an embodiment.

FIG. 5 is a flowchart of a method by which the medical imaging apparatus processes a medical image, according to an embodiment.

FIG. 6 illustrates a second scout image of an object displayed through a user interface by the medical imaging apparatus, according to an embodiment.

FIG. 7 illustrates a second scout image of an object displayed through a user interface by the medical imaging apparatus, according to an embodiment.

FIG. 8A illustrates a first scout image of an object displayed through a user interface by the medical imaging apparatus, according to an embodiment.

FIG. 8B illustrates a second scout image of the object displayed through the user interface by the medical imaging apparatus, according to an embodiment.

FIG. 8C illustrates a second scout image of the object displayed through the user interface by the medical imaging apparatus, according to an embodiment.

FIG. 9 is a schematic diagram of a general MRI system.

### MODE OF DISCLOSURE

The present specification describes principles of the present disclosure and sets forth embodiments thereof to clarify the scope of the present disclosure and to allow one of ordinary skill in the art to implement the embodiments. The present embodiments may have different forms.

Like reference numerals refer to like elements throughout. The present specification does not describe all components in the embodiments, and common knowledge in the art or the same descriptions of the embodiments will be omitted below. The term “part” or “portion” may be implemented using hardware or software, and according to embodiments, one “part” or “portion” may be formed as a single unit or element or include a plurality of units or elements. Hereinafter, the principles and embodiments of the present disclosure will be described in detail with reference to the accompanying drawings.

In the present specification, an “image” may include a medical image obtained by a medical imaging apparatus such as a magnetic resonance imaging (MRI) apparatus, a computed tomography (CT) apparatus, an ultrasound imaging apparatus, or an X-ray apparatus.

In the present specification, an “object” may be a target to be imaged and include a human, an animal, or a part of a human or animal. For example, the object may include a body part (an organ) or a phantom.

An MRI system acquires an MR signal and reconstructs the acquired MR signal into an image. The MR signal denotes a radio frequency (RF) signal emitted from the object.

In the MRI system, a main magnet creates a static magnetic field to align a magnetic dipole moment of a specific atomic nucleus of the object placed in the static magnetic field along a direction of the static magnetic field. A gradient coil may generate a gradient magnetic field by applying a gradient signal to a static magnetic field and induce resonance frequencies differently according to each region of the object.

An RF coil may emit an RF signal to match a resonance frequency of a region of the object whose image is to be acquired. Furthermore, when gradient magnetic fields are applied, the RF coil may receive MR signals having different resonance frequencies emitted from a plurality of regions of the object. Though this process, the MRI system may obtain an image from an MR signal by using an image reconstruction technique.

As used herein, the term “and/or” includes any and all combinations of one or more of the associated listed items. Expressions such as “at least one of”, when preceding a list of elements, modify the entire list of elements and do not modify the individual elements of the list.

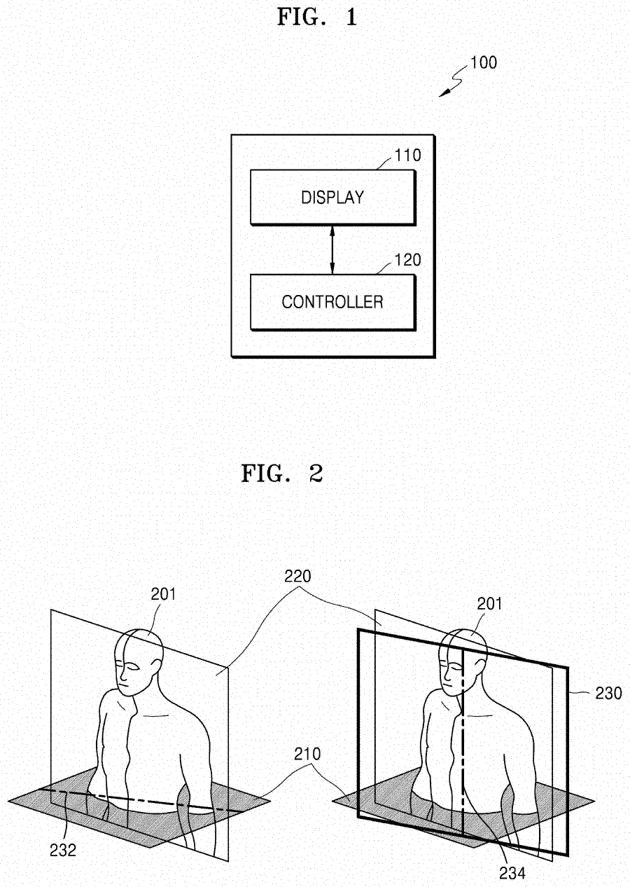

FIG. 1 is a block diagram of a medical imaging apparatus 100 according to an embodiment.

Referring to FIG. 1, the medical imaging apparatus 100 includes a display 110 and a controller 120.

The medical imaging apparatus 100 may provide a user interface including information needed to obtain a medical image of an object. The medical imaging apparatus 100 may display a scout image for medical imaging planning of the object through the user interface and may provide information about a position of a region of interest.

The medical imaging apparatus 100 may be a medical apparatus such as an ultrasound imaging apparatus, a CT apparatus, or an MRI apparatus. Alternatively, the medical imaging apparatus 100 may be included in a medical apparatus such as a CT apparatus, an MRI apparatus, or an X-ray apparatus, or may be connected to a medical apparatus.

The following will be explained on the assumption that the medical imaging apparatus 100 is an MRI apparatus for processing a magnetic resonance (MR) image.

The display 110 may display the user interface for displaying the scout image and receiving an input that selects the region of interest.

The display 110 may display an image of the object through the user interface. The image of the object may be a scout image that enables a user to select a section of interest. Alternatively, the image of the object may be a final medical image of the section of interest.

The controller 120 may obtain an MR signal based on a pulse sequence stored in a memory (not shown) of the medical imaging apparatus 100 or a pulse sequence received from an external device (not shown). For example, the MR signal may be a signal received from a scanner (not shown). Alternatively, the MR signal may be received from the memory of the medical imaging apparatus 100 or the external device (not shown).

The controller 120 may obtain volume data by processing the obtained MR signal. Also, the controller 120 may be implemented in various ways according to a type of the medical imaging apparatus 100. When the medical imaging apparatus 100 according to an embodiment is included in or connected to a CT apparatus, the controller 120 may obtain the volume data by receiving and processing X-rays passing through the object.

The controller 120 may obtain an MR image of the object, based on the obtained volume data of the object. The controller 120 may include a module for reconstructing the obtained MR image.

The MR image obtained by the controller 120 may be a scout image obtained by using a scout scan. According to the scout scan, the user may select the section of interest through the scout image before the final image is obtained. The user may be an operator who captures a medical image by using the medical imaging apparatus 100.

The controller 20 may obtain a plurality of scout images corresponding to planes that are perpendicular to one another.

For example, the scout images corresponding to the planes that are perpendicular to one another may be images in an axial view, a sagittal view, and a coronal view.

The controller 120 may receive an input that selects the region of interest in a first scout image corresponding to a first plane from among the plurality of scout images. The controller 120 may receive a user input that selects the region of interest.

The user input may be an input that selects the region of interest in the first scout image of the user interface displayed on the display 110.

The controller 120 may display a first quadrangle indicating a third plane that is a plane corresponding to the region of interest in a second scout image corresponding to a second plane from among the plurality of scout images, based on the input that selects the region of interest.

The first plane and the second plane may be perpendicular to one another.

For example, the first plane may correspond to an axial view, and the second plane may correspond to a sagittal view. In contrast, the first plane may correspond to a sagittal view, and the second plane may correspond to an axial view. Alternatively, the first plane may correspond to an axial view, and the second plane may correspond to a coronal view. In contrast, the first plane may correspond to a coronal view, and the second plane may correspond to an axial view.

According to an embodiment, the first plane may be perpendicular to the third plane.

The controller 120 may display the first quadrangle so that at least a portion of the first quadrangle has a fade effect.

The fade effect may depict a level of darkness corresponding to a distance from the second plane. Due to the fade effect, the controller 120 may display the first quadrangle indicating the third pane to provide depth perception. In this case, since the controller 120 three-dimensionally displays the first quadrangle, the user may easily understand that the first quadrangle indicates the third plane.

Also, the controller 120 may display the first quadrangle indicating the third plane so that the first quadrangle overlaps a position a position corresponding to the region of interest in the second scout image corresponding to the second plane.

According to an embodiment, the controller 120 may display, in a reduced shape, a second quadrangle corresponding to the first quadrangle on an outer portion of the second scout image.

The controller 120 may display the second quadrangle so that at least one side of the second quadrangle includes a guide line depicting depth perception.

The controller 120 may display the first quadrangle so that at least one side of the first quadrangle includes a guide line depicting depth perception. When at least one side of a quadrangle includes a guide line depicting depth perception, the quadrangle may be displayed in a trapezoidal shape.

Also, the controller 120 may display a line of intersection between the second plane and the third plane on the second scout image.

The controller 120 may display the first quadrangle so that only a portion closer to a point of view of the second scout image than the second plane has the fade effect.

The controller 120 may display a first line indicating the region of interest included in the first scout image. The first line may correspond to a line of intersection between the first plane and the third plane.

The controller 120 may obtain an MR image of the region of interest. The display 110 may display the obtained MR image.

Since the medical imaging apparatus 100 of FIG. 1 displays the second scout image and the first quadrangle, the user may easily understand a position of the region of interest in a space including the object.

FIG. 2 is a cross-sectional view for explaining images obtained by the medical imaging apparatus 100 according to an embodiment.

Referring to FIG. 2, the medical imaging apparatus 100 may obtain scout images corresponding to a plurality of planes, by using a scout scan before an image of a region of interest of an object 201 is obtained.

For example, the medical imaging apparatus 100 may obtain scout images corresponding to a first plane 210 and a second plane 220.

The first plane 210 of FIG. 2 may correspond to an axial view, and the second plane 220 may correspond to a coronal view. Also, the first plane 210 may correspond to an axial view, and the second plane 220 may correspond to a sagittal view. The first plane 210 and the second plane 220 of FIG. 2 are exemplarily illustrated, and planes are not limited thereto.

Also, referring to FIG. 2, a third plane 230 may be a plane corresponding to the region of interest. A first line 232 may correspond to a line of intersection between the first plane 210 and the third plane 230. Also, a second line 234 may correspond to a line of intersection between the second plane 220 and the third plane 230.

The medical imaging apparatus 100 may receive a user input that selects the region of interest corresponding to the third plane 230, and then may obtain a final image of the region of interest based on the user input.

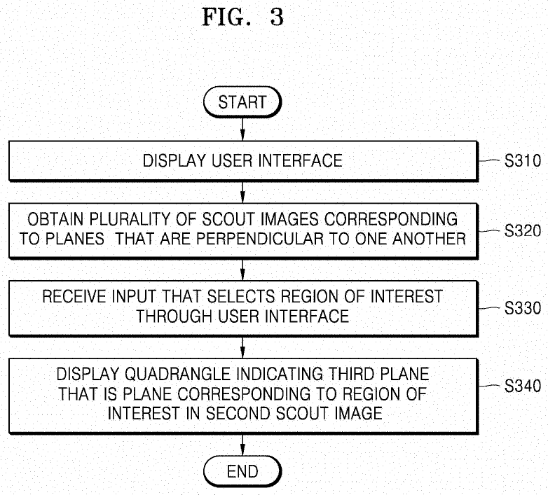

FIG. 3 is a flowchart of a method by which the medical imaging apparatus 100 processes a medical image according to an embodiment.

In operation S310, the medical imaging apparatus 100 may display a user interface.

In operation S320, the medical imaging apparatus 100 may obtain a plurality of scout images corresponding to planes that are perpendicular to one another.

In operation S330, the medical imaging apparatus 100 may receive an input that selects a region of interest through the user interface. The user interface may include an image corresponding to a first plane from among the plurality of scout images.

In operation S340, the medical imaging apparatus 100 may display a first quadrangle indicating a third plane that is a plane corresponding to the region of interest in a second scout image.

According to an embodiment, the medical imaging apparatus 100 may display a quadrangle indicating the third plane in the second scout image based on an input that selects the region of interest received through the user interface. Also, the medical imaging apparatus 100 may display the first quadrangle so that at least a portion of the first quadrangle has a fade effect. The fade effect may depict a level of darkness corresponding to a distance from a second plane.

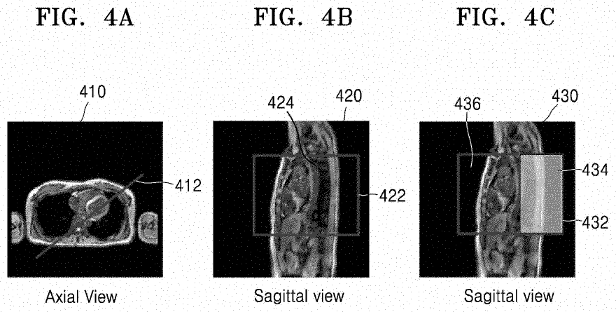

FIG. 4A illustrates a first scout image 410 of an object displayed through a user interface by the medical imaging apparatus 100 according to an embodiment.

The first scout image 410 of FIG. 4A may correspond to a first plane. Also, the first scout image 410 may correspond to an axial view.

The medical imaging apparatus 100 may receive an input that selects a region of interest through the user interface including the first scout image 410.

For example, the medical imaging apparatus 100 may receive a user input that selects the region of interest by generating a geometric figure indicating the region of interest in the first scout image 410 through the user interface or changing a position of the geometric figure indicating the region of interest. Examples of the geometric figure displayed in a place corresponding to the region of interest may include a line and a quadrangle, and there is no limitation in a shape of the geometric figure for indicating the place corresponding to the region of interest.

Referring to FIG. 4A, the medical imaging apparatus 100 may display a first line 412 in the place corresponding to the region of interest.

FIG. 4B illustrates a second scout image 420 of the object displayed through the user interface by the medical imaging apparatus 100 according to an embodiment.

The second scout image 420 of FIG. 4B may correspond to a second plane. Also, the second scout image 420 may correspond to a sagittal view.

The medical imaging apparatus 100 may display a first quadrangle 422 indicating a third plane that is a plane corresponding to the region of interest in the second scout image 420.

For example, the first quadrangle 422 of FIG. 4B may indicate a plane corresponding to the first line 412 of FIG. 4A.

The medical imaging apparatus 100 may display a line of intersection between the second plane and the third plane as a second line 4254.

FIG. 4C illustrates a second scout image 430 of the object displayed through the user interface by the medical imaging apparatus 100 according to an embodiment.

The second scout image 430 of FIG. 4C may correspond to a second plane. Also, the second scout image 430 may correspond to a sagittal view. Referring to FIG. 4C, the medical imaging apparatus 100 may display a first quadrangle indicating a third plane that is a plane corresponding to the region of interest so that a portion 434 of the first quadrangle 432 has a fade effect.

The fade effect may depict a level of darkness corresponding to a distance from the second plane. For example, the medical imaging apparatus 100 may display the first quadrangle 432 so that a portion of the first quadrangle 432 indicating an area far from the second plane is darker than other portions.

For example, the medical imaging apparatus 100 may display the first quadrangle 432 so that only the portion 434 indicating a place closer to a point of view of the second scout image 430 than the second plane has the fade effect and other portions 436 do not have the fade effect. Since the medical imaging apparatus 100 displays the first quadrangle 432 in the second scout image 430 with the fade effect, spatial understanding of a user may be improved.

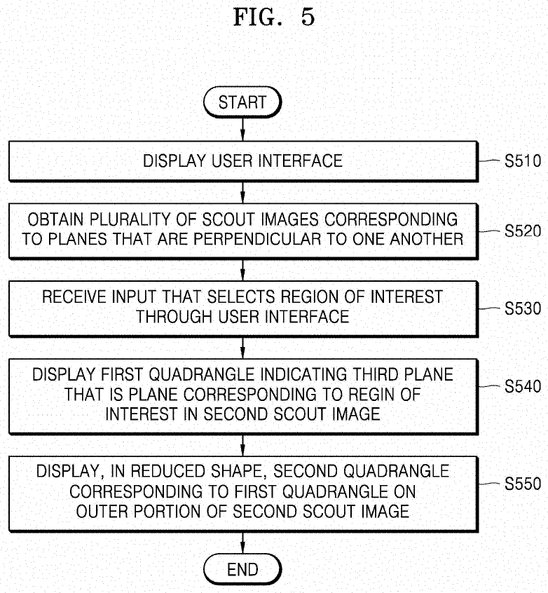

FIG. 5 is a flowchart of a method by which the medical imaging apparatus 100 processes a medical image according to an embodiment.

In operation S510, the medical imaging apparatus 100 may display a user interface.

In operation S520, the medical imaging apparatus 100 may obtain a plurality of scout images corresponding to planes that are perpendicular to one another.

In operation S530, the medical imaging apparatus 100 may receive an input that selects a region of interest through the user interface. The user interface may include an image corresponding to a first plane from among the plurality of scout images.

In operation S540, the medical imaging apparatus 100 may display a first quadrangle indicating a third plane that is a plane corresponding to the region of interest in a second scout image.

In operation S550, the medical imaging apparatus 100 may display, in a reduced shape, a second quadrangle corresponding to the first quadrangle on an outer portion of the second scout image.

The medical imaging apparatus 100 according to an embodiment may display the second quadrangle so that at least a portion of the second quadrangle has a fade effect. Also, the medical imaging apparatus 100 may display the second quadrangle so that at least one side of the second quadrangle includes a guide line depicting depth perception.

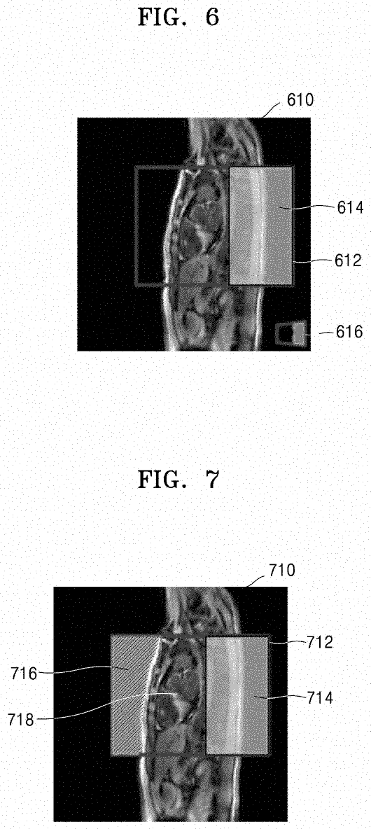

FIG. 6 illustrates a second scout image 610 of an object displayed through a user interface by the medical imaging apparatus 100 according to an embodiment.

The second scout image 610 of FIG. 6 may correspond to a second plane.

Referring to FIG. 6, the medical imaging apparatus 100 may display a first quadrangle 612 indicating a third plane in the second scout image 610. The third plane may be a plane corresponding to a region of interest. The medical imaging apparatus 100 may display the first quadrangle 612 so that a portion 614 of the first quadrangle 612 has a fade effect.

The medical imaging apparatus 100 may display, in a reduced shape, a second quadrangle 616 corresponding to the first quadrangle 612 on an outer portion of the second scout image 610. The medical imaging apparatus 100 may display the second quadrangle 616 to provide depth perception of the second quadrangle 616.

When the second quadrangle 616 is displayed to provide depth perception of the second quadrangle 616, it may mean that at least one side of the second quadrangle 616 includes a guide line depicting depth perception. For example, the guide line depicting depth perception may include two opposite sides of the second quadrangle 616.

A user may intuitively recognize a positional relationship between the third plane corresponding to the first quadrangle 612 and the second plane corresponding to the second scout image 610, through the second quadrangle 616.

FIG. 7 illustrates a second scout image 710 of an object displayed through a user interface by the medical imaging apparatus 100 according to an embodiment.

The second scout image 710 of FIG. 7 may correspond to a second plane.

Referring to FIG. 7, the medical imaging apparatus 100 may display a first quadrangle 712 indicating a third plane in the second scout image 710. The third plane may be a plane corresponding to a region of interest.

The medical imaging apparatus 100 may display the first quadrangle 712 so that a first portion 714 of the first quadrangle 712 has a fade effect. Also, the medical imaging apparatus 100 may display the first quadrangle 712 so that a second portion 716 of the first quadrangle 712 has a fade effect. Also, the medical imaging apparatus 100 may display the first quadrangle 712 so that a third portion 718 of the first quadrangle 712 does not have a fade effect.

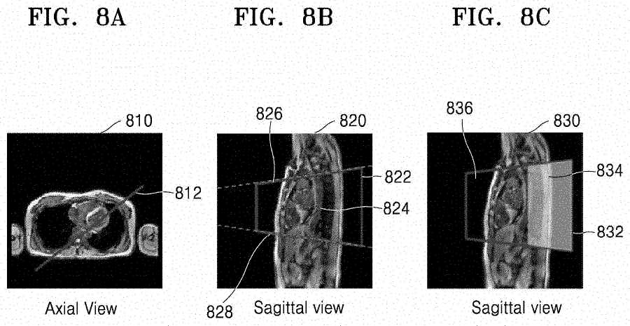

FIG. 8A illustrates a first scout image 810 of an object displayed through a user interface by the medical imaging apparatus 100 according to an embodiment.

The first scout image 810 of FIG. 8A may correspond to a first plane and may correspond to an axial view. The medical imaging apparatus 100 may receive an input that selects a region of interest through the user interface including the first scout image 810.

Referring to FIG. 8A, the medical imaging apparatus 100 may display a first line 812 in a place corresponding to a region of interest.

FIG. 8B illustrates a second scout image 820 of the object displayed through the user interface by the medical imaging apparatus 100 according to an embodiment.

The second scout image 820 of FIG. 8B may correspond to a second plane and may correspond to a sagittal view.

The medical imaging apparatus 100 may display a first quadrangle 822 indicating a third plane that is a plane corresponding to the region of interest in the second scout image 820.

For example, the first quadrangle 822 of FIG. 8B may indicate a plane corresponding to the first line 812 of FIG. 8A.

The medical imaging apparatus 100 may display a line of intersection between the second plane and the third plane as a second line 824.

The medical imaging apparatus 100 may display the first quadrangle 822 to provide depth perception. In detail, the medical imaging apparatus 100 may display the first quadrangle 822 so that at least one side of the first quadrangle 822 includes a guide line depicting depth perception. The guide line depicting depth perception may include two opposite sides of the first quadrangle 822. For example, referring to FIG. 8B, the medical imaging apparatus 100 may display the first quadrangle 822 so that a first side 826 and a second side 828 of the first quadrangle 822 include a guide line depicting depth perception.

FIG. 8C illustrates a second scout image 830 of the object displayed through the user interface by the medical imaging apparatus 100 according to an embodiment.

The second scout image 830 of FIG. 8C may correspond to a second plane, and may correspond to a sagittal view. Referring to FIG. 8C, the medical imaging apparatus 100 may display the first quadrangle 822 indicating a third plane that is a plane corresponding to the region of interest so that a portion 834 of the first quadrangle 822 has a fade effect.

For example, the medical imaging apparatus 100 may display the first quadrangle 832 so that a portion indicating a place far from the second plane in the first quadrangle 832 is darker than other portions.

For example, the medical imaging apparatus 100 may display the first quadrangle 832 so that only the portion 834 indicating a place closer to a point of view of the second scout image 830 than the second plane has the fade effect and other portions 836 do not have the fade effect. The medical imaging apparatus 100 may enable a user to feel a 3D effect due to the fade effect.

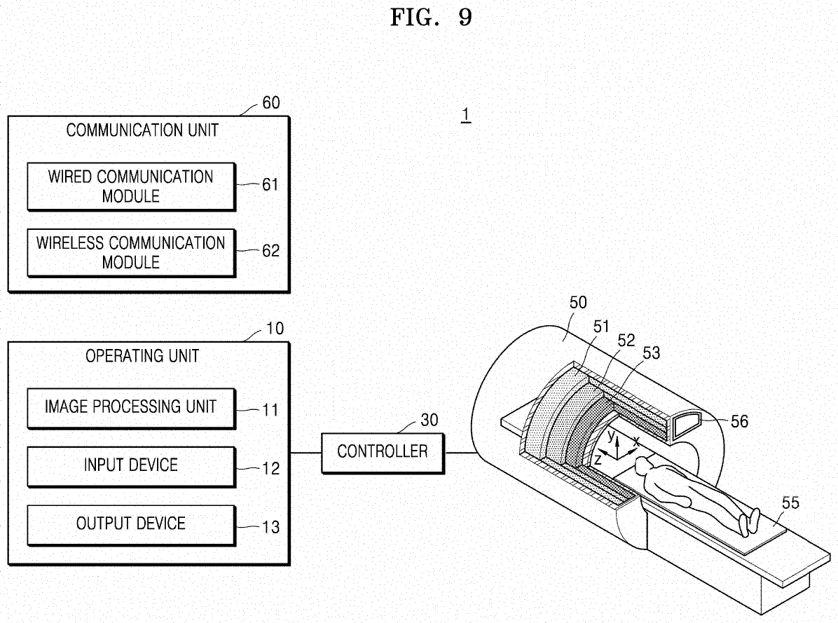

FIG. 9 is a schematic diagram of an MRI system 1.

Referring to FIG. 9, the MRI system 1 may include an operating unit 10, a controller 30, and a scanner 50. The controller 30 may be independently implemented as shown in FIG. 9. Alternatively, the controller 30 may be separated into a plurality of sub-components and incorporated into elements of the MRI system 1. Each component in the MRI system 1 will now be described in detail.

The scanner 50 may be formed to have a cylindrical shape (e.g., a shape of a bore) having an empty inner space into which an object may be inserted. A static magnetic field and a gradient magnetic field are created in the inner space of the scanner 50, and an RF signal is emitted toward the inner space.

The scanner 50 may include a static magnetic field generator 51, a gradient magnetic field generator 52, an RF coil unit 53, a table 55, and a display 56. The static magnetic field generator 51 creates a static magnetic field for aligning magnetic dipole moments of atomic nuclei of the object in a direction of the static magnetic field. The static magnetic field generator 51 may be formed as a permanent magnet or superconducting magnet using a cooling coil.

The gradient magnetic field generator 52 is connected to the controller 30 and generates a gradient magnetic field by applying a gradient to a static magnetic field in response to a control signal received from the controller 30. The gradient magnetic field generator 52 includes X, Y, and Z coils for generating gradient magnetic fields in X-, Y-, and Z-axis directions crossing each other at right angles and generates a gradient signal according to a position of a region being imaged so as to differently induce resonance frequencies according to regions of the object.

The RF coil unit 53 connected to the controller 30 may emit an RF signal toward the object in response to a control signal received from the controller 30 and receive an MR signal emitted from the object. In detail, the RF coil unit 53 may transmit, toward atomic nuclei of the object having precessional motion, an RF signal having the same frequency as that of the precessional motion, stop transmitting the RF signal, and then receive an MR signal emitted from the object.

The RF coil unit 53 may be formed as a transmitting RF coil for generating an electromagnetic wave having an RF corresponding to the type of an atomic nucleus, a receiving RF coil for receiving an electromagnetic wave emitted from an atomic nucleus, or one transmitting/receiving RF coil serving both functions of the transmitting RF coil and receiving RF coil. Furthermore, in addition to the RF coil unit 53, a separate coil may be attached to the object. Examples of the separate coil may include a head coil, a spine coil, a torso coil, and a knee coil according to a region being imaged or to which the separate coil is attached.

The display 56 may be disposed outside and/or inside the scanner 50. The display 56 is also controlled by the controller 30 to provide a user or the object with information related to medical imaging.

The display 56 may include the display 110 of FIG. 1.

Furthermore, the scanner 50 may include an object monitoring information acquisition unit (not shown) configured to acquire and transmit monitoring information about a state of the object. For example, the object monitoring information acquisition unit may acquire monitoring information related to the object from a camera (not shown) for capturing images of a movement or position of the object, a respiration measurer (not shown) for measuring the respiration of the object, an ECG measurer for measuring the electrical activity of the heart, or a temperature measurer for measuring a temperature of the object and transmit the acquired monitoring information to the controller 30. The controller 30 may in turn control an operation of the scanner 50 based on the monitoring information. The controller 30 will now be described in more detail.

The controller 30 may control overall operations of the scanner 50.

The controller 30 may control a sequence of signals formed in the scanner 50. The controller 30 may control the gradient magnetic field generator 52 and the RF coil unit 53 according to a pulse sequence received from the operating unit 10 or a designed pulse sequence.

A pulse sequence may include all pieces of information required to control the gradient magnetic field generator 52 and the RF coil unit 53. For example, the pulse sequence may include information about a strength, a duration, and application timing of a pulse signal applied to the gradient magnetic field generator 52.

The controller 30 may control a waveform generator (not shown) for generating a gradient wave, i.e., an electrical pulse according to a pulse sequence and a gradient amplifier (not shown) for amplifying the generated electrical pulse and transmitting the same to the gradient magnetic field generator 52. Thus, the controller 30 may control formation of a gradient magnetic field by the gradient magnetic field generator 52.

The controller 30 may control an operation of the RF coil unit 53. For example, the controller 30 may supply an RF pulse having a resonance frequency to the RF coil unit 30 that emits an RF signal toward the object, and receive an MR signal received by the RF control unit 53. In this case, the controller 30 may adjust emission of an RF signal and reception of an MR signal according to an operating mode by controlling an operation of a switch (e.g., a T/R switch) for adjusting transmitting and receiving directions of the RF signal and the MR signal based on a control signal.

The controller 30 may control a movement of the table 55 where the object is placed. Before imaging is performed, the controller 30 may move the table 55 according to which region of the object is to be imaged.

The controller 30 may also control the display 56. For example, the controller 30 control the on/off state of the display 56 or a screen to be output on the display 56 according to a control signal.

The controller 30 may be formed as an algorithm for controlling operations of the components in the MRI system 1, a memory (not shown) for storing data in the form of a program, and a processor for performing the above-described operations by using the data stored in the memory. In this case, the memory and the processor may be implemented as separate chips. Alternatively, the memory and processor may be incorporated into a single chip.

The controller 30 may include the controller 120 of FIG. 1.

The operating unit 10 may control overall operations of the MRI system 1 and include an image processing unit 11, an input device 12, and an output device 13.

The image processing unit 11 may control the memory to store an MR signal received from the controller 30, and generate image data with respect to the object from the stored MR signal by applying an image reconstruction technique by using an image processor.

For example, if a k space (for example, also referred to as a Fourier space or a frequency space) of the memory is filled with digital data to complete k-space data, the image processing unit 11 may reconstruct image data from the k-space data by applying various image reconstruction techniques (e.g., by performing inverse Fourier transform on the k-space data) by using the image processor.

Furthermore, the image processing unit 11 may perform various signal processing operations on MR signals in parallel. For example, the image processing unit 11 may perform signal processing on a plurality of MR signals received via a multi-channel RF coil in parallel so as to convert the plurality MR signals into image data. In addition, the image processing unit 11 may store the image data in the memory, or the controller 30 may store the same in an external server via a communication unit 60 as will be described below.

The input device 12 may receive, from the user, a control command for controlling the overall operations of the MRI system 1. For example, the input device 12 may receive, from the user, object information, parameter information, a scan condition, and information about a pulse sequence. The input device 12 may be a keyboard, a mouse, a track ball, a voice recognizer, a gesture recognizer, a touch screen, or any other input device.

The output device 13 may output image data generated by the image processing unit 11. The output device 13 may also output a user interface (UI) configured so that the user may input a control command related to the MRI system 1. The output device 13 may be formed as a speaker, a printer, a display, or any other output device.

Furthermore, although FIG. 9 shows that the operating unit 10 and the controller 30 are separate components, the operating unit 10 and the controller 30 may be included in a single device as described above. Furthermore, processes respectively performed by the operating unit 10 and the controller 30 may be performed by another component. For example, the image processing unit 11 may convert an MR signal received from the controller 30 into a digital signal, or the controller 30 may directly perform the conversion of the MR signal into the digital signal.

The MRI system 1 may further include the communication unit 60 and may be connected to an external device (not shown) such as a server, a medical apparatus, and a portable device (e.g., a smartphone, a tablet PC, a wearable device, etc.) via the communication unit 60.

The communication unit 60 may include at least one component that enables communication with an external device. For example, the communication unit 60 may include at least one of a local area communication module (not shown), a wired communication module 61, and a wireless communication module 62.

The communication unit 60 may receive a control signal and data from an external device and transmit the received control signal to the controller 30 so that the controller 30 may control the MRI system 1 according to the received control signal.

Alternatively, by transmitting a control signal to an external device via the communication unit 60, the controller 30 may control the external device according to the control signal.

For example, the external device may process data of the external device according to a control signal received from the controller 30 via the communication unit 60.

A program for controlling the MRI system 1 may be installed on the external device and may include instructions for performing some or all of the operations of the controller 30.

The program may be preinstalled on the external device, or a user of the external device may download the program from a server providing an application for installation. The server providing an application may include a recording medium having the program recorded thereon.

Embodiments may be implemented through non-transitory computer-readable recording media having recorded thereon computer-executable instructions and data. The instructions may be stored in the form of program codes, and when executed by a processor, generate a predetermined program module to perform a specific operation. Furthermore, when being executed by the processor, the instructions may perform specific operations according to the embodiments.

While one or more embodiments have been described with reference to the figures, it will be understood by one of ordinary skill in the art that various changes in form and details may be made therein without departing from the spirit and scope of the present disclosure as defined by the following claims. Accordingly, the above embodiments and all aspects thereof are examples only and are not limiting.

1. A medical imaging apparatus comprising:
a display configured to display a user interface; and
a controller configured to
obtain a plurality of scout images corresponding to planes that are perpendicular to one another,
receive an input that selects, through the user interface, a region of interest in a first scout image corresponding to a first plane from among the plurality of scout images, and
display, in a second scout image corresponding to a second plane from among the plurality of scout images, a first quadrangle indicating a third plane that is a plane corresponding to the region of interest, based on the input,
wherein the controller is further configured to display the first quadrangle so that at least a portion of the first quadrangle has a fade effect,
wherein the fade effect depicts a level of darkness corresponding to a distance from the second plane.

2. The medical imaging apparatus of claim 1, wherein the controller is further configured to display, in a reduced shape, a second quadrangle corresponding to the first quadrangle on an outer portion of the second scout image.

3. The medical imaging apparatus of claim 2, wherein the controller is further configured to display the second quadrangle so that at least one side of the second quadrangle comprises a guide line depicting depth perception.

4. The medical imaging apparatus of claim 1, wherein the controller is further configured to:
display the first quadrangle so that the first quadrangle overlaps a position indicating the region of interest in the second scout image,
display a line of intersection between the second plane and the third plane on the second scout image; and
display the first quadrangle so that only a portion closer to a point of view of the second scout image than the second plane has the fade effect.

5. The medical imaging apparatus of claim 1, wherein the controller is further configured to display the first quadrangle so that at least one side of the first quadrangle comprises a guide line depicting depth perception.

6. The medical imaging apparatus of claim 1, wherein the controller is further configured to display a first line indicating the region of interest included in the first scout image,
wherein the first line corresponds to a line of intersection between the first plane and the third plane.

7. The medical imaging apparatus of claim 1, wherein the first plane is perpendicular to the third plane.

8. The medical imaging apparatus of claim 1, wherein each of the plurality of scout images corresponds to one from among an axial view, a sagittal view, and a coronal view.

9. The medical imaging apparatus of claim 1, wherein the controller is further configured to obtain a magnetic resonance (MR) image of the region of interest.

10. A method of processing a medical image, the method comprising:
displaying a user interface;
obtaining a plurality of scout images corresponding to planes that are perpendicular to one another;
receiving an input that selects a region of interest through the user interface comprising a first scout image corresponding to a first plane from among the plurality of scout images; and
displaying, in a second scout image corresponding to a second plane from among the plurality of scout images, a first quadrangle indicating a third plane that is a plane corresponding to the region of interest, based on the input,
wherein the first quadrangle is displayed so that at least a portion of the first quadrangle has a fade effect,
wherein the fade effect depicts a level of darkness corresponding to a distance from the second plane.

11. The method of claim 10, wherein the displaying comprises displaying, in a reduced shape, a second quadrangle corresponding to the first quadrangle on an outer portion of the second scout image.

12. The method of claim 11, wherein the displaying comprises displaying the second quadrangle so that at least one side of the second quadrangle comprises a guide line depicting depth perception.

13. The method of claim 10, wherein the displaying comprises:
displaying the first quadrangle so that the first quadrangle overlaps a position indicating the region of interest in the second scout image;
displaying a line of intersection between the second plane and the third plane on the second scout image; and
displaying the first quadrangle so that only a portion closer to a point of view of the second scout image than the second plane has the fade effect.

14. The method of claim 10, wherein the displaying comprises displaying the first quadrangle so that at least one side of the first quadrangle comprises a guide line depicting depth perception.

15. The method of claim 10, wherein the displaying comprises displaying a first line indicating the region of interest included in the first scout image,
wherein the first line corresponds to a line of intersection between the first plane and the third plane.
